(19) United States
(12) Patent Application Publication
(10) Pub. No.: US 2015/0217070 A1
(43) Pub. Date: Aug. 6, 2015

(54) CONNECTOR OF DOUBLE-LUMEN TUBE FOR DIFFERENTIAL LUNG VENTILATION

(71) Applicant: Insung Medical Co., Ltd., Seoul (KR)

(72) Inventors: Junho Song, Seoul (KR); Taejin Kang, Ansan-si (KR)

(21) Appl. No.: 14/484,283

(22) Filed: Sep. 12, 2014

(30) Foreign Application Priority Data
Feb. 6, 2014 (KR) 10-2014-0013865

(51) Int. Cl. A61M 16/04; A61M 16/20; A61M 16/00; A61M 16/08

(52) U.S. Cl. CPC A61M 16/0404; A61M 16/0833; A61M 16/0486; A61M 16/201; A61M 16/0875; A61M 16/0057; A61M 16/209; A61M 2210/1035; A61M 2205/583

(57) ABSTRACT

A connector of a double-lumen tube for Differential Lung Ventilation is provided. The connector includes a tube connection unit disposed at one side of the double-lumen tube, and branched in one direction to connect to the ventilator, with the pair of lumen tubes of the double-lumen tube inserted into the tube connection unit, a connection valve unit disposed in the one direction in which the tube connection unit is branched, for controlling a flow amount of gas or fluid provided to the respective branched lumen tubes and externally indicating an operation state of a valve that controls the flow amount of the gas or the fluid, and a ventilator connection unit disposed at one side of the connection valve unit, in connection to the ventilator, for providing pressure of the ventilator separately to the pair of lumen tubes through the connection valve unit.

This application claims the benefit of Korean Patent Application No. 10-2014-0013865, filed on Feb. 6, 2014, which is hereby incorporated by reference as if fully set forth herein.

### BACKGROUND OF THE INVENTION

1. Field of the Invention

The present invention relates to a connector of a double-lumen tube for Differential Lung Ventilation (DLV), and more particularly, to a connector of a double-lumen tube for DLV, which increases manipulation convenience and safety by enabling easy pressure-regulated flow control through a user manipulation in ventilating the lungs separately.

2. Discussion of the Related Art

DLV is a technique to ventilate either the right lung or the left lung separately. For DLV, a double-lumen tube with two combined tubes intubated into the separate lungs is used as an endotracheal tube. DLV is useful when it is necessary to separate one lung from the other lung due to a lung operation or infection, bleeding, etc. of the one lung. In the case where the right and left lungs are placed in different pathological conditions, DLV is also considered to be appropriate in ventilating the lungs in their respective local conditions.

During an operation of one lung or an operation of an important part near to a lung, for example, the heart, it is necessary to secure an unobstructed field of vision. The lungs are a passive organ that moves during respiration. The lungs can be forced to stop by blocking air entry into the lungs by means of a double-lumen tube.

The afore-mentioned double-lumen tube has a pair of tubes joined together. The two tubes are configured so as to be branched into the bronchi connected to the lungs respectively through the mouth and then the airway to thereby separately ventilate the lungs, when the double-lumen tube is intubated into a patient.

To perform DLV using the double-lumen tube, with the double-lumen tube intubated into a patient, a ventilator that provides suction pressure or discharge pressure is connected to the pair of tubes by means of a connector and the lungs of the patient are ventilated separately according to the states of the lungs and a surgical method.

Although the ventilator is typically a device that supplies air to the lungs, it may be used as a device that injects fluid or gas such as oxygen or anesthetic gas into the lungs to treat the patient.

Conventionally, a connector of a double-lumen tube for DLV is inserted into respective branched pipes that connect a ventilator to the double-lumen tube and applies pressure of the ventilator or supplies fluid or gas such as oxygen or anesthetic gas from the ventilator directly to the double-lumen tube.

However, the conventional connector for a double-lumen tube is designed just to transfer pressure generated from the ventilator. Therefore, it is difficult to block pressure, fluid, or gas such as oxygen or anesthetic gas immediately during operation or treatment and much time is taken to do so.

If on-going supply of pressure from the ventilator is abruptly stopped, the remaining internal pressure still applies to a certain degree even after blocking the pressure. The resulting application of the pressure to the lungs may affect the treatment or operation.

Also, if on-going supply of fluid or gas from the ventilator is abruptly stopped, the remaining fluid or gas inside a supply pipe may be supplied to the lungs, thus affecting the treatment or operation.

Particularly, considering that DLV is a process of providing different levels of pressure to the lungs during operation or treatment in the case of disease in a lung or a bad lung state, the life of a patient may be at risk unless an immediate medical action is taken for the patient. Accordingly, if pressure is blocked to the lungs during DLV based on a medical decision, immediate pressure blocking is difficult by manipulating the ventilator. Moreover, continuous application of the remaining pressure to the lungs may affect the treatment or operation, thus increasing the risk.

In general, the connector is clamped by means of forceps with non-serrated blades to immediately block fluid or gas to a lung.

The use of the forceps with non-serrated blades means that the fluid or gas is completely blocked, which is rather risky. With the complete blocking of the fluid or gas, only one lung functions, thereby causing hypoxia and thus placing a patient in danger.

### SUMMARY OF THE INVENTION

Accordingly, the present invention is directed to a connector of a double-lumen tube for differential lung ventilation that substantially obviates one or more problems due to limitations and disadvantages of the related art.

An object of the present invention is to provide a connector of a double-lumen tube for Differential Lung Ventilation (DLV), which is configured to connect the double-lumen tube injected to apply pressure separately to both lungs to a ventilator that provides pressure, in a manner that enables immediate pressure-regulated flow control through a user manipulation, thus increasing the stability of DLV and manipulation convenience.

Additional advantages, objects, and features of the invention will be set forth in part in the description which follows and in part will become apparent to those having ordinary skill in the art upon examination of the following or may be learned from practice of the invention. The objectives and other advantages of the invention may be realized and attained by the structure particularly pointed out in the written description and claims hereof as well as the appended drawings.

To achieve these objects and other advantages and in accordance with the purpose of the invention, as embodied and broadly described herein, a connector of a double-lumen tube for DLV, for connecting a pair of lumen tubes of the double-lumen tube intubated respectively into both bronchi through an airway to a ventilator that provides pressure includes a tube connection unit disposed at one side of the double-lumen tube, and branched in one direction to connect to the ventilator, with the pair of lumen tubes of the double-lumen tube inserted into the tube connection unit, a connection valve unit disposed in the one direction in which the tube connection unit is branched, for controlling a flow amount of gas or fluid provided to the respective branched lumen tubes and externally indicating an operation state of a valve that controls the flow amount of the gas or the fluid, and a ventilator connection unit disposed at one side of the connection valve unit, in connection to the ventilator, for providing pressure of the ventilator separately to the pair of lumen tubes through the connection valve unit.

The tube connection unit may include a tube connection body disposed at one side of the double-lumen tube and having one portion branched through the connection valve unit and the other portion into which the double-lumen tube is inserted, a first branched pipe disposed at one side of the tube connection body and installed at a position branched through the connection valve unit, for connection to one of the pair of lumen tubes when the double-lumen tube is connected, and a second branched pipe disposed at the one side of the tube connection body and installed at a position branched through the connection valve unit, for connection to the other lumen tube when the double-lumen tube is connected.

The connection valve unit may include a connection valve body installed in the one direction in which the tube connection unit is branched and including a first valve space communicating with one of the pair of lumen tubes, at a branched position of the tube connection unit and a second valve space communicating with the other lumen tube, at a branched position of the tube connection unit, a first rotation valve disposed in one direction from a center of the connection valve body, installed rotatably in the first valve space, and including a first circulation space at a position connected to the one lumen tube, for circulating the gas or the fluid supplied from the ventilator and a first valve knob protruding in one direction to allow a user to grab the first valve knob, for controlling the flow amount of the gas or the fluid to the one lumen tube by controlling an area over which the first circulation space is opened and closed, and a second rotation valve disposed in the other direction from the center of the connection valve body, installed rotatably in the second valve space, and including a second circulation space at a position connected to the other lumen tube, for circulating the gas or the fluid supplied from the ventilator and a second valve knob protruding in one direction to allow the user to grab the second valve knob, for controlling the flow amount of the gas or the fluid to the other lumen tube by controlling an area over which the second circulation space is opened and closed.

The connector may further include a reference position indicator provided at the center of the connection valve body and including protruding scales between the first and second rotational valves, for indicating a reference position for adjusting the first and second rotational valves that rotate, a first position indicator disposed at a position of the second rotational valve in the other direction from the first rotation valve, with a plurality of scales directed toward the reference position indicator, the scales apart from one another by a predetermined distance, for indicating a position to which the flow amount of the gas or the fluid is controlled with respect to the reference position indicator according to a degree of opening or closing of the first circulation space, and a second position indicator disposed at a position of the first rotation valve in one direction from the second rotation valve, with a plurality of scales directed toward the reference position indicator, the scales apart from one another by a predetermined distance, for indicating a position to which the flow amount of the gas or the fluid is controlled with respect to the reference position indicator according to a degree of opening or closing of the second circulation space.

The ventilator connection unit may include a first ventilator connection body disposed at the one side of the connection valve unit connected to one of the pair of lumen tubes and having one lateral end connected to the ventilator at a lateral position, a first connection body disposed at the other side of the first ventilator connection body, for connecting the first ventilator connection body to the connection valve unit connected to the one lumen tube, a second ventilator connection body disposed at the one side of the connection valve unit connected to the other lumen tube and having one lateral end connected to the ventilator at a lateral position, and a second connection body disposed at the other side of the second ventilator connection body, for connecting the second ventilator connection body to the connection valve unit connected to the other lumen tube.

The connector may further include a first drain body disposed at the other lateral end of the first ventilator connection body, for opening or closing the other lateral end of the first ventilator connection body to drain internal pressure through a user manipulation, and a second drain body disposed at the other lateral end of the second ventilator connection body, for opening or closing the other lateral end of the second ventilator connection body to drain internal pressure through a user manipulation.

It is to be understood that both the foregoing general description and the following detailed description of the present invention are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this application, illustrate embodiment(s) of the invention and together with the description serve to explain the principle of the invention. In the drawings:

FIG. 1 is a perspective view of a connector of a double-lumen tube for Differential Lung Ventilation (DLV) according to an embodiment of the present invention;

FIG. 2 is a front view of the connector of a double-lumen tube for DLV illustrated in FIG. 1;

FIG. 3 is a right side view illustrating a use state with a first valve body closed in the connector of a double-lumen tube for DLV illustrated in FIG. 1;

FIG. 4 is a right side view illustrating a use state with the first valve body open in the connector of a double-lumen tube for DLV illustrated in FIG. 1;

FIG. 5 is a left side view illustrating a use state with a second valve body open and a second drain body open in the connector of a double-lumen tube for DLV illustrated in FIG. 1;

FIG. 6 is a right side view illustrating a use state with a first valve body closed in a connector of a double-lumen tube for DLV according to another embodiment of the present invention;

FIG. 7 is a right side view illustrating a use state with the first valve body open in the connector of a double-lumen tube for DLV illustrated in FIG. 6;

FIG. 8 is a left side view illustrating a use state with a second valve body closed in the connector of a double-lumen tube for DLV illustrated in FIG. 6;

FIG. 9 is a perspective view of a connector of a double-lumen tube for DLV according to another embodiment of the present invention;

FIG. 10 is a right side view of the connector of a double-lumen tube for DLV illustrated in FIG. 9;

FIG. 11 is a view illustrating a use state with a connection valve unit completely open in the connector of a double-lumen tube for DLV illustrated in FIG. 9;

FIG. 12 is a view illustrating a use state with a part of the connection valve unit closed, as a first rotation valve is operated in the use state of the connector of a double-lumen tube for DLV illustrated in FIG. 11; and

FIG. 13 is a view illustrating a use state with the connection valve unit completely closed, as the first rotation valve is operated in the use state of the connector of a double-lumen tube for DLV illustrated in FIG. 12.

### DETAILED DESCRIPTION OF THE INVENTION

Reference will now be made in detail to the preferred embodiments of the present invention with reference to the accompanying drawings, so that those skilled in the art can implement the present invention readily.

Techniques which are well known to the technical field of the present invention and have no direct relation to the present invention are omitted lest they should obscure the subject matter of the invention.

Some components are shown exaggerated, omitted, or shown schematically in the attached drawings for the same reason. Also, each component is not shown in a size matching to a real size. The same or corresponding components are denoted by like reference numerals in the drawings.

With reference to the attached drawings, a connector of a double-lumen tube for Differential Lung Ventilation (DLV) according to embodiments of the present invention will be described below in detail.

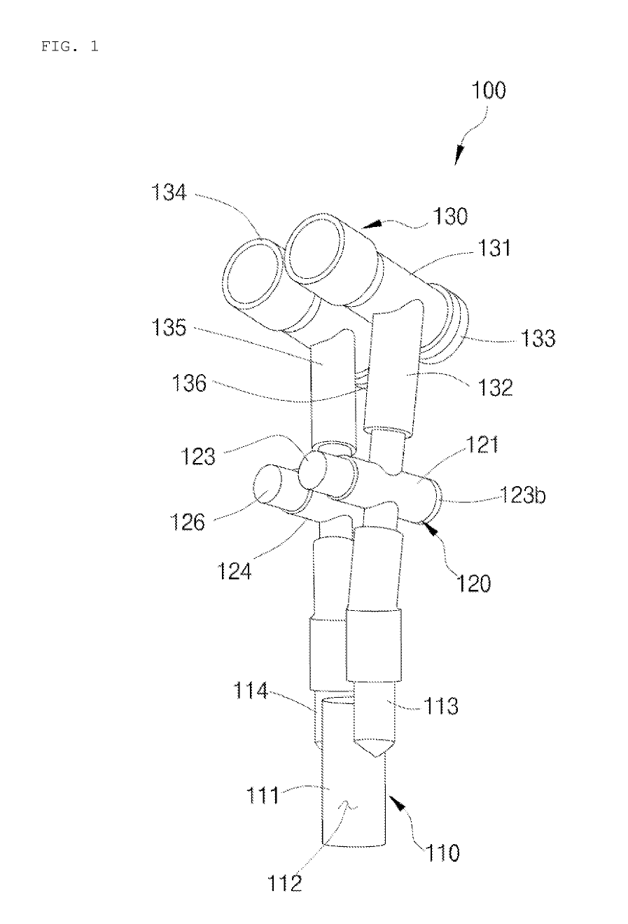

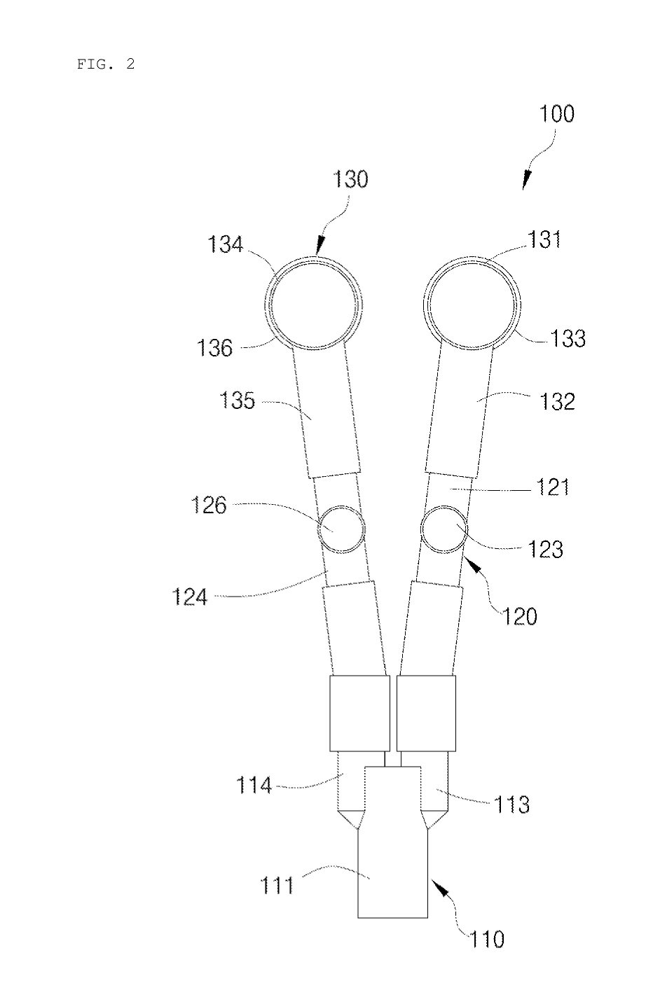

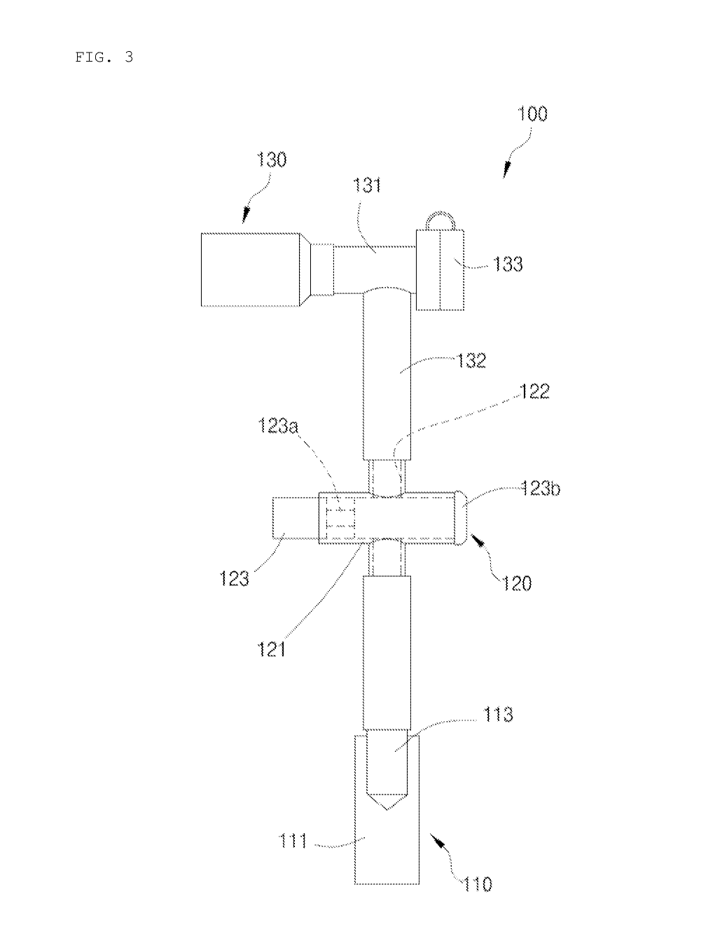

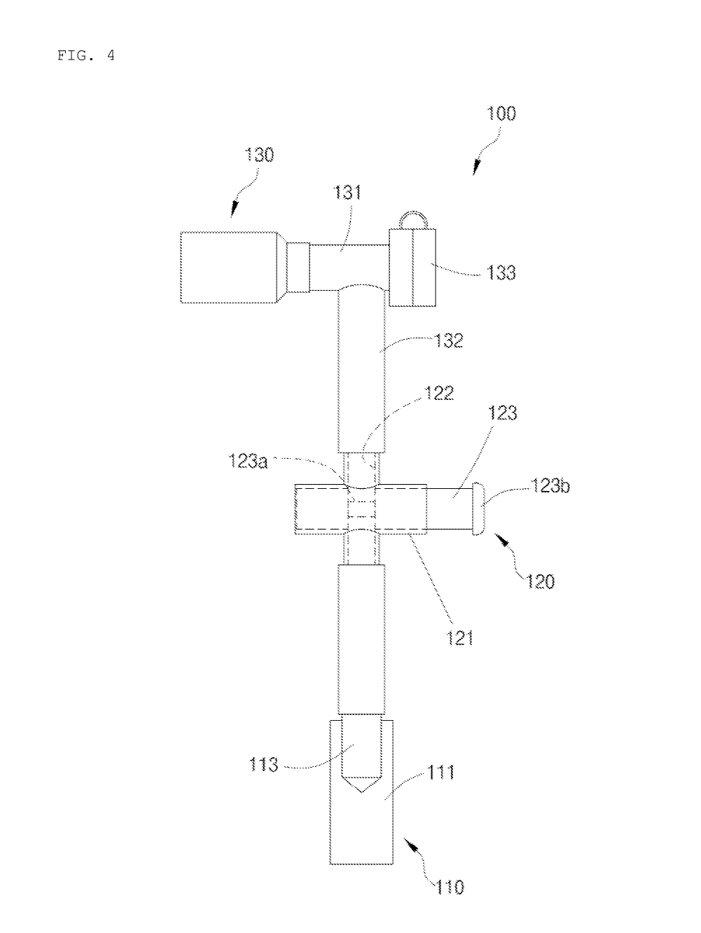

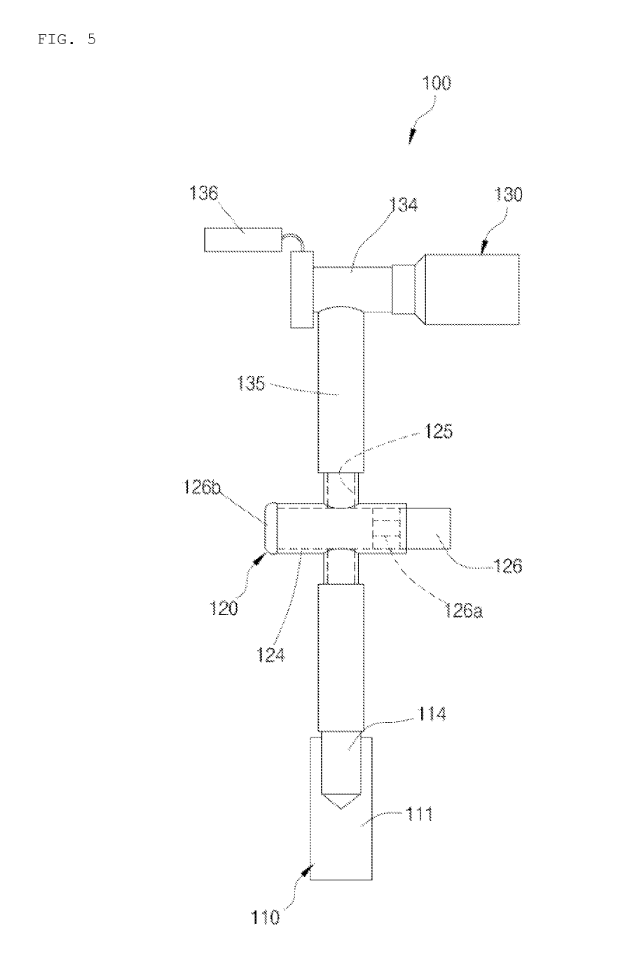

FIG. 1 is a perspective view of a connector of a double-lumen tube for DLV according to an embodiment of the present invention, FIG. 2 is a front view of the connector of a double-lumen tube for DLV illustrated in FIG. 1, FIG. 3 is a right side view illustrating a use state with a first valve body closed in the connector of a double-lumen tube for DLV illustrated in FIG. 1, FIG. 4 is a right side view of a use state with the first valve body open in the connector of a double-lumen tube for DLV illustrated in FIG. 1, and FIG. 5 is a left side view of a use state with a second valve body open and a second drain body open in the connector of a double-lumen tube for DLV illustrated in FIG. 1.

Referring to FIGS. 1 to 5, a connector 100 of a double-lumen tube for DLV according to an embodiment of the present invention is a device that connects a double-lumen tube (not shown) including a pair of tubes intubated respectively into the bronchi through the airway to a ventilator (not shown) that provides pressure.

To ventilate the lungs separately, the double-lumen tube includes a pair of tubes which are inserted into the right and left lungs respectively through the airway and is connected to the ventilator by means of the connector 100, for receiving suction pressure or discharge pressure from the ventilator.

The connector 100 of a double-lumen tube for DLV is connected to the pair of tubes inserted separately into the right and left lungs and to the ventilator that provides pressure to the respective lungs.

To describe a direction in which the connector 100 is connected to the double-lumen tube with the pair of tubes inserted respectively into the right and left lungs, one of two parts is selectively referred to as a “first” one and the other unselected part is referred to as a “second” one, to thereby distinguish them from each other.

A direction from the bronchi communicating with the lungs to the airway is called “one” direction (portion, end, or side) and a direction from the airway to the bronchi is called “the other” direction (portion, end, or side). A position at a right angle with the one direction (portion, end, or side) and the other direction (portion, end, or side) is called “lateral”. In this manner, directionality is defined to thereby describe an accurate position and operation direction of each component.

The connector 100 of a double-lumen tube for DLV includes a tube connection unit 110 connected to the double-lumen tube, a valve unit 120, and a ventilator connection unit 130.

The tube connection unit 110 includes a tube connection body 111 into which one portions of a pair of lumen tubes are inserted, a first branched pipe 113, and a second branched pipe 114. The tube connection body 111 is disposed at one side of the double-lumen tube and includes a tube connection space 112 into which the pair of lumen tubes are inserted, when the double-lumen tube is connected to the connector 100. The tube connection space 112 has one end opened and the other end communicating with the first branched pipe 113 and the second branched pipe 114, inside the tube connection body 111. When the tube connection body 111 is connected to the double-lumen tube, the pair of lumen tubes are inserted together into the tube connection body 111 from one direction to the other direction so that the pair of lumen tubes may be connected respectively to the first and second branched pipes 113 and 114.

The first branched pipe 113 is disposed at one side of the tube connection body 111, and installed at a branched position for connection to one of the two lumen tubes, when the double-lumen tube is connected to the tube connection body 111. The first branched pipe 113 is branched to a position for communication with one portion of the tube connection space 112 according to a position where the first branched pipe 113 is connected to one of the lumen tubes inserted into the right or left lung. Thus, the first branched pipe 113 may provide ventilation pressure to the connected lumen tube.

The second branched pipe 114 is disposed at the one side of the tube connection body 111, and installed at a branched position for connection to the other lumen tube opposite to the one lumen tube, when the double-lumen tube is connected to the tube connection body 111. The second branched pipe 114 is branched to a position opposite to the first branched pipe 113, for example, to the left if the first branched pipe 113 is disposed to the right or to the right if the first branched pipe 113 is disposed to the left, for communication with the one portion of the tube connection space 112. Thus, the second branched pipe 114 may provide ventilation pressure to the other lumen tube.

The valve unit 120 includes a first valve body 121 and a first stopper 123 provided at the first branched pipe 113, and a second valve body 124 and a second stopper 126 provided at the second branched pipe 114. The first valve body 121 is disposed at one side of the first branched pipe 113 and connected to the first branched pipe 113 connected to the one lumen tube. Thus, when the double-lumen tube is connected to the connector 100, the first valve body 121 transfers or blocks pressure provided by the ventilator to the one lumen tube through the first branched pipe 113, at the position where the first valve body 121 is connected to the first branched pipe 113.

A first circulation space 122 penetrates through the first valve body 121 from one side to the other side of the first valve body 121 in order to transfer pressure from the ventilator. The first circulation space 122 is formed to penetrate through the first valve body 121 from one direction to the other direction so that it may provide pressure of the ventilator connected to the ventilator connection unit 130 to the first branched pipe 113.

The first stopper 123 is disposed along a lateral direction, movably through a center of the first valve body 121. The first stopper 123 includes a first transfer groove 123a as a groove designed to open the first circulation space 122. As the first stopper 123 moves in the lateral direction through a user manipulation, the first stopper 123 may close or open the first circulation space 122. The first stopper 123 is installed to be movable in the first valve body 121. If the first stopper 123 moves in one lateral direction with respect to the center of the first valve body 121, the first stopper 123 blocks the first circulation space 122. As the first transfer groove 123a is formed in the other lateral direction, for air circulation, if the first stopper 123 moves in the other lateral direction, it opens the first circulation space 122, for pressure transfer.

The valve unit 120 further includes a first catching body 123b protruding radially from one lateral end of the first stopper 123, for being caught over the first valve body 121 and thus preventing slide-off of the first stopper 123 when the first stopper 123 moves to block the first circulation space 122.

That is, when the first circulation space 122 is to be blocked in the first valve body 121, the user moves the first stopper 123 in the other lateral direction, blocking the first circulation space 122, while the first catching body 123b is caught and thus prevents the first stopper 123 from sliding off. When the first circulation space 122 is to be opened in the first valve body 121, the user moves the first stopper 123 in the one lateral direction, so that the first transfer groove 123a may be positioned in the first circulation space 122 and thus may transfer pressure.

The second valve body 124 is disposed at one side of the second branched pipe 114 and connected to the second branched pipe 114 connected to the other lumen tube. Thus, when the double-lumen tube is connected to the connector 100, the second valve body 121 transfers or blocks pressure provided by the ventilator to the other lumen tube through the second branched pipe 114, at the position where the second valve body 124 is connected to the second branched pipe 114.

A second circulation space 125 penetrates through the second valve body 124 from one side to the other side of the second valve body 124 in order to transfer pressure from the ventilator. The second circulation space 125 is formed to penetrate through the second valve body 124 from one direction to the other direction so that it may provide pressure of the ventilator connected to the ventilator connection unit 130 to the second branched pipe 114.

The second stopper 126 is disposed along a lateral direction, movably through a center of the second valve body 124. The second stopper 126 includes a second transfer groove 126a as a groove designed to open the second circulation space 125. As the second stopper 126 moves in the lateral direction through a user manipulation, the second stopper 126 may close or open the second circulation space 125. The second stopper 126 is installed to be movable in the second valve body 124. If the second stopper 126 moves in one lateral direction with respect to the center of the second valve body 124, the second stopper 126 blocks the second circulation space 125. As the second transfer groove 126a is formed in the other lateral direction, for air circulation, if the second stopper 126 moves in the other lateral direction, it opens the second circulation space 125, for pressure transfer.

The valve unit 120 further includes a second catching body 126b protruding radially from one lateral end of the second stopper 126, for being caught over the second valve body 124 and thus preventing slide-off of the second stopper 126 when the second stopper 126 moves to block the second circulation space 125.

That is, when the second circulation space 125 is to be blocked in the second valve body 124, the user moves the second stopper 126 in the other lateral direction, blocking the second circulation space 125, while the second catching body 126b is caught and thus prevents the second stopper 126 from sliding off. When the second circulation space 125 is to be opened in the second valve body 121, the user moves the second stopper 126 in the one lateral direction, so that the second transfer groove 126a may be positioned in the second circulation space 125 and thus may transfer pressure.

The ventilator connection unit 130 includes a first ventilator connection body 131, a first connection body 132, and a first drain body 133 which are installed at one side of the first valve body 121, and a second ventilator connection body 134, a second connection body 135, and a second drain body 136 which are installed at one side of the second valve body 124. The first ventilator connection body 131 is disposed at the one side of the first valve body 121 connected to the one lumen tube and has one lateral end connected to the ventilator, at a lateral position. As the first ventilator connection body 131 is at the right angle with a direction in which the first valve body 121 is connected to the one lumen tube, the first ventilator connection body 131 is easily connected to the ventilator.

The first connection body 132 is disposed at the other side of the first ventilator connection body 131, connecting the first ventilator connection body 131 to the first valve body 121 connected to the one lumen tube. As the first connection body 132 connects the first ventilator connection body 131 connected to the ventilator to the first valve body 121 connected to the one lumen tube, the first connection body 132 transfers pressure of the ventilator under the control of the first valve body 121.

The first drain body 133 is disposed at the other lateral end of the first ventilator connection body 131 so as to open or close the other lateral end of the first ventilator connection body 131 and thus drain internal pressure through a user manipulation. When pressure of the ventilator is transferred to one portion of the one lumen tube connected to the first branched pipe 113 through the first ventilator connection body 131 under the control of the first valve body 121, the first drain body 133 keeps the first ventilator connection body 131 closed. Also, when the first valve body 121 is closed or the ventilator applies excess pressure, the first drain body 133 is opened and drains the internal pressure of the first ventilator connection body 131.

That is, when the first valve body 121 is blocked or excess pressure is provided by the ventilator, the first drain body 133 drains the internal pressure of the first ventilator connection body 131. Particularly, if pulsation occurs, that is, pressure is not uniformly generated from the ventilator, unintended pressure may be provided to a lung, thereby causing a problem in the lung. Then, immediate draining may increase stability.

While the user attaches or removes the first drain body 133 to open or close the first ventilator connection body 131, this is purely exemplary. Accordingly, it is apparent to those skilled in the art that any means is available as the first drain body 133 as far as it can open or close the other lateral end of the first ventilator connection body 131.

The second ventilator connection body 134 is disposed at the one side of the second valve body 124 connected to the other lumen tube and has one lateral end connected to the ventilator, at a lateral position. As the second ventilator connection body 134 is at the right angle with a direction in which the second valve body 124 is connected to the other lumen tube, the second ventilator connection body 134 is easily connected to the ventilator.

The second connection body 135 is disposed at the other side of the second ventilator connection body 134, connecting the second ventilator connection body 134 to the second valve body 124 connected to the other lumen tube. As the second connection body 135 connects the second ventilator connection body 134 connected to the ventilator to the second valve body 124 connected to the other lumen tube, the second connection body 135 transfers pressure of the ventilator under the control of the second valve body 124.

The second drain body 136 is disposed at the other lateral end of the second ventilator connection body 134 so as to open or close the other lateral end of the second ventilator connection body 134 and thus drain internal pressure through a user manipulation. When pressure of the ventilator is transferred to one portion of the other lumen tube connected to the second branched pipe 114 through the second ventilator connection body 134 under the control of the second valve body 124, the second drain body 136 keeps the second ventilator connection body 134 closed. Also, when the second valve body 124 is closed or the ventilator applies excess pressure, the second drain body 136 is opened and drains the internal pressure of the second ventilator connection body 134.

Particularly, if pulsation occurs, that is, pressure is not uniformly generated from the ventilator, unintended pressure may be provided to a lung, thereby causing a problem in the lung. Then, immediate draining may increase stability.

While the user attaches or removes the second drain body 136 to open or close the second ventilator connection body 134, this is purely exemplary. Accordingly, it is apparent to those skilled in the art that any means is available as the second drain body 136 as far as it can open or close the other lateral end of the second ventilator connection body 134.

Now a description will be given of a connector of a double-lumen tube for DLV according to another embodiment of the present invention.

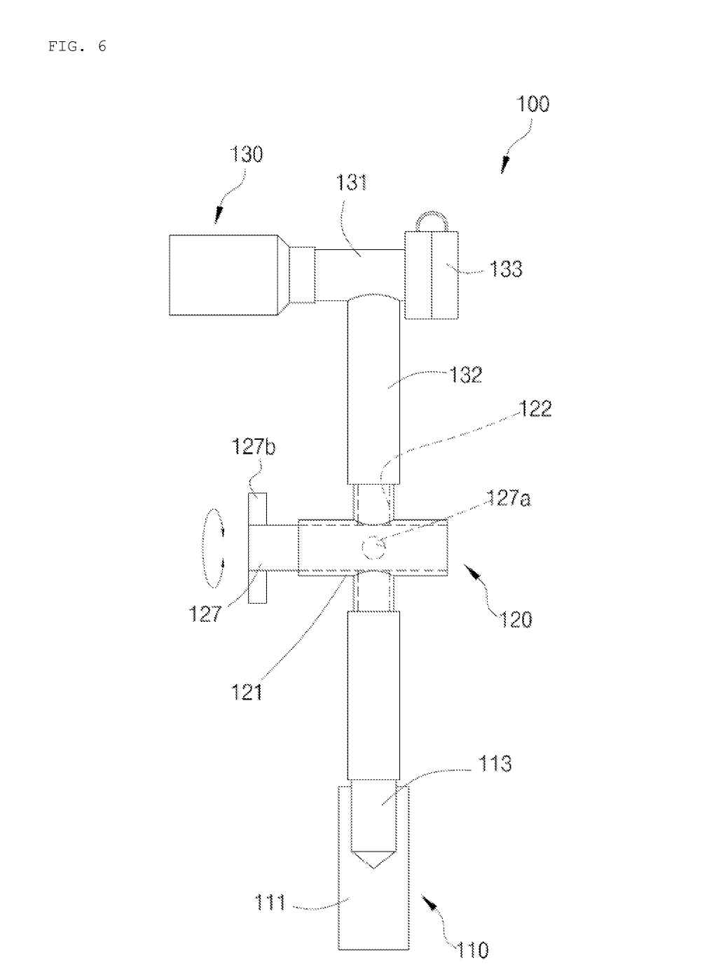

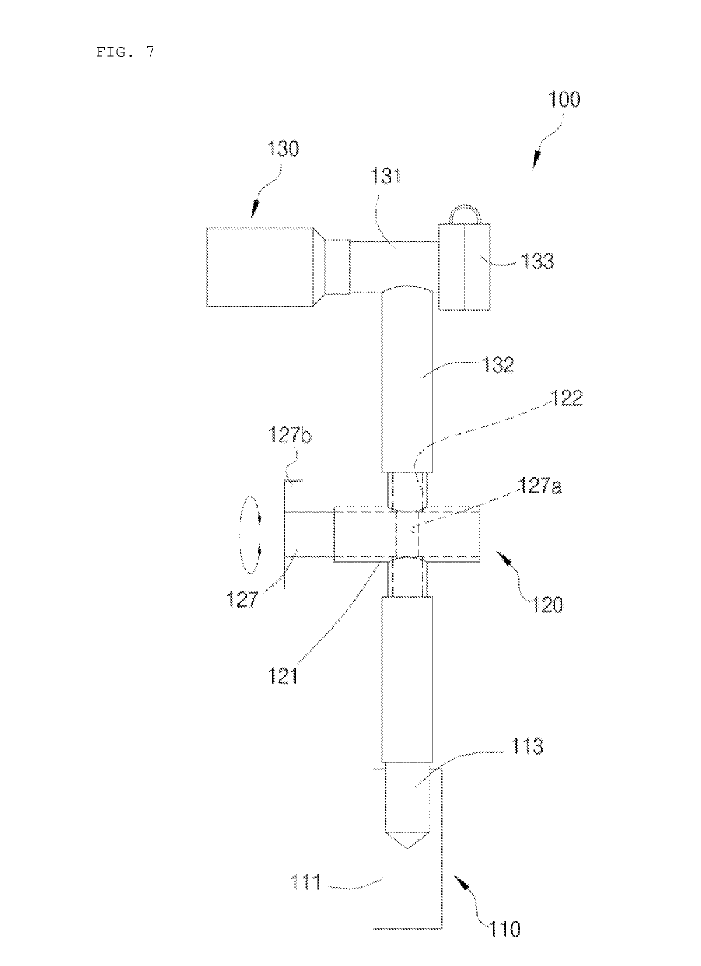

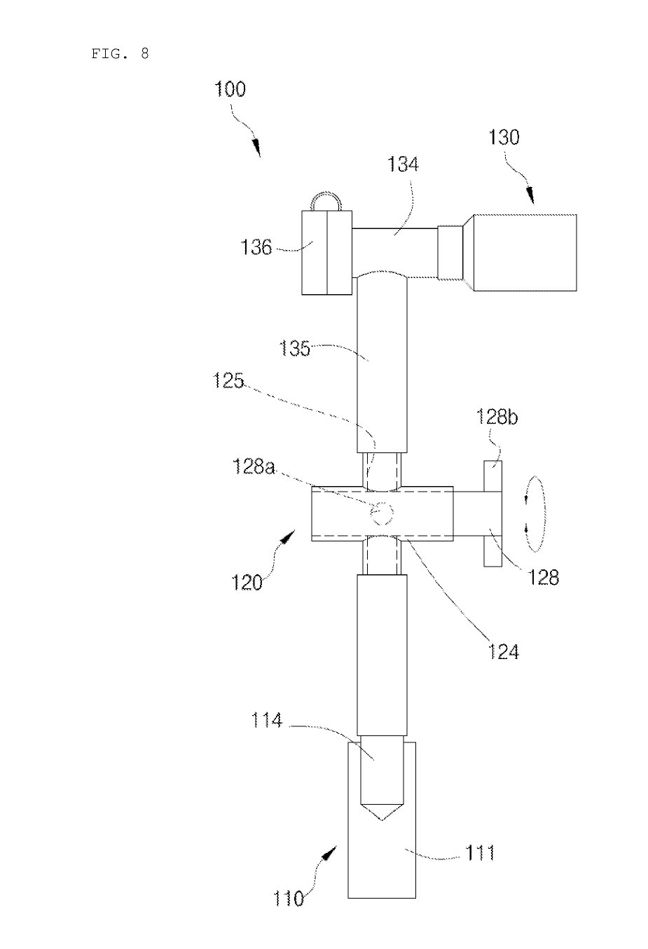

FIG. 6 is a right side view illustrating a use state with a first valve body closed in a connector of a double-lumen tube for DLV according to another embodiment of the present invention, FIG. 7 is a right side view illustrating a use state with the first valve body open in the connector of a double-lumen tube for DLV illustrated in FIG. 6, and FIG. 8 is a left side view illustrating a use state with a second valve body closed in the connector of a double-lumen tube for DLV illustrated in FIG. 6.

Referring to FIGS. 6, 7, and 8, the connector 100 of a double-lumen tube for DLV according to another embodiment of the present invention includes the tube connection unit 110, the valve unit 120, and the ventilator connection unit 130. Some components of the tube connection unit 110, the valve unit 120, and the ventilator connection unit 130 are identical to their counterparts in the connector 100 of a double-lumen tube for DLV illustrated in FIG. 1 to and thus will not be described herein. The following description centers on a configuration of the valve unit 120 different from in the connector 100 of a double-lumen tube for DLV illustrated in FIGS. 1 to 5.

The valve unit 120 includes the first valve body 121, the second valve body 124, a first rotation stopper 127, and a second rotation stopper 128. The first and second valve bodies 121 and 124 are identical to those of the valve unit 120 illustrated in FIGS. 1 to 5 and thus their description is not provided herein.

The first rotation stopper 127 is disposed along a lateral direction, rotatably at the center of the first valve body 121. The first rotation stopper 127 rotates to thereby selectively open or block the first circulation space 122.

A first rotational circulation space 127a is formed to penetrate at a position corresponding to the first circulation space 122 in the first rotation stopper 127 so that the first rotation stopper 127 may rotate to selectively open or block the first circulation space 122. If the user rotates the first rotation stopper 127 and thus the first circulation space 122 communicates with the first rotational circulation space 127a, the first circulation space 122 is opened. If the first circulation space 122 is brought to a position at which it does not communicate with the first rotational circulation space 127a along with rotation of the first rotation stopper 127, the first circulation space 122 is closed.

A first rotation knob 127b is provided at one lateral end of the first rotation stopper 127 so that the user may rotate the first rotation stopper 127 by grabbing the first rotation knob 127b.

The second rotation stopper 128 is disposed along a lateral direction, rotatably at the center of the second valve body 124. The second rotation stopper 128 rotates to thereby selectively open or block the second circulation space 125.

A second rotational circulation space 128a is formed to penetrate at a position corresponding to the second circulation space 125 in the second rotation stopper 128 so that the second rotation stopper 128 may rotate to selectively open or block the second circulation space 125. If the user rotates the second rotation stopper 128 and thus the second circulation space 125 communicates with the second rotational circulation space 128a, the second circulation space 125 is opened. If the second circulation space 125 is brought to a position at which it does not communicate with the second rotational circulation space 128a along with rotation of the second rotation stopper 128, the second circulation space 125 is closed.

A second rotation knob 128b is provided at one lateral end of the second rotation stopper 128 so that the user may rotate the second rotation stopper 128 by grabbing the second rotation knob 128b.

Accordingly, as the user selectively open or block the first and second valve bodies 121 and 124 by rotating the first and second rotation stoppers 127 and 128, ventilation pressure that the ventilator provides to the double-lumen tube may be controlled.

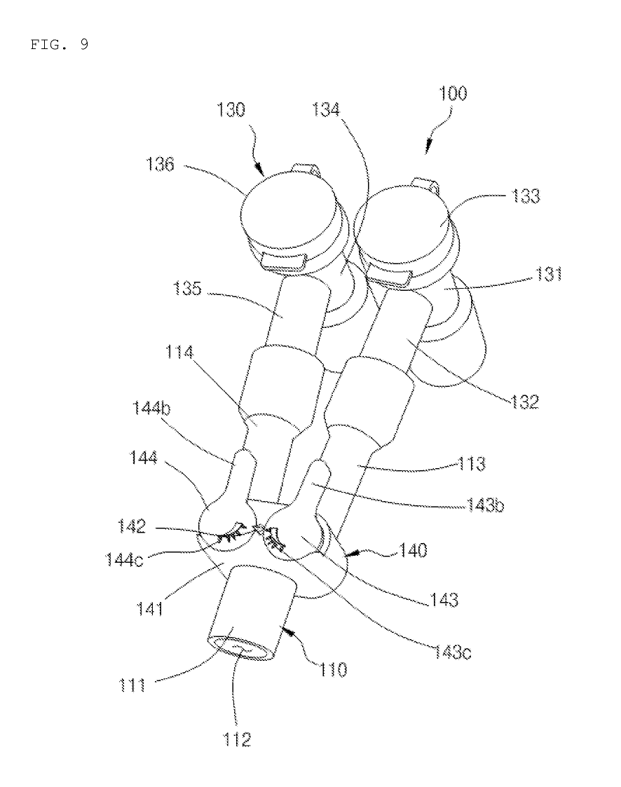

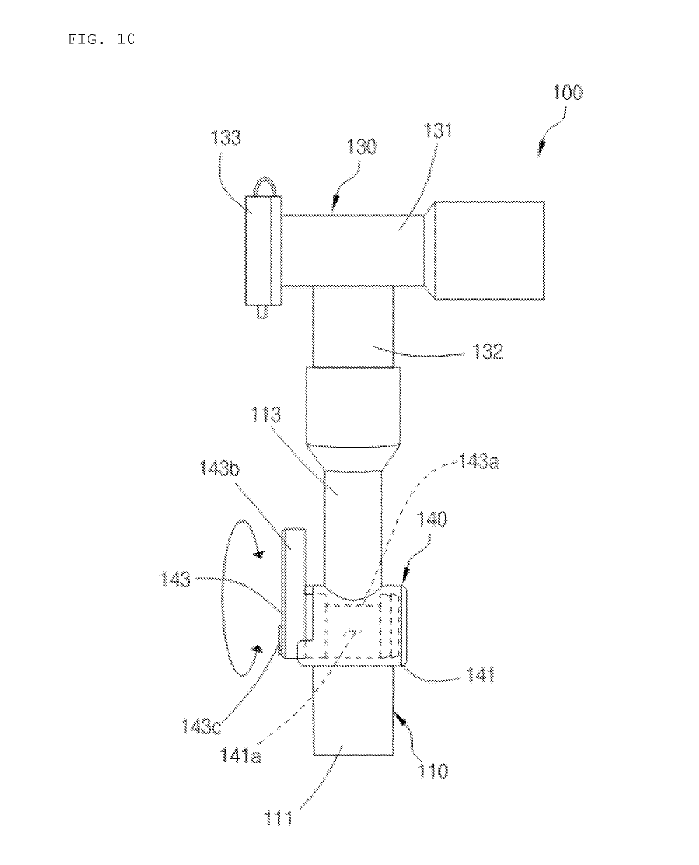

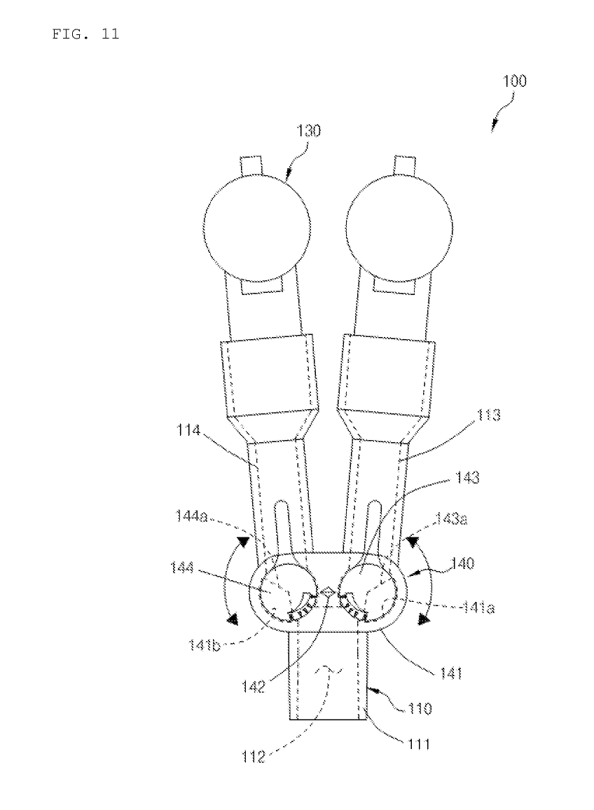

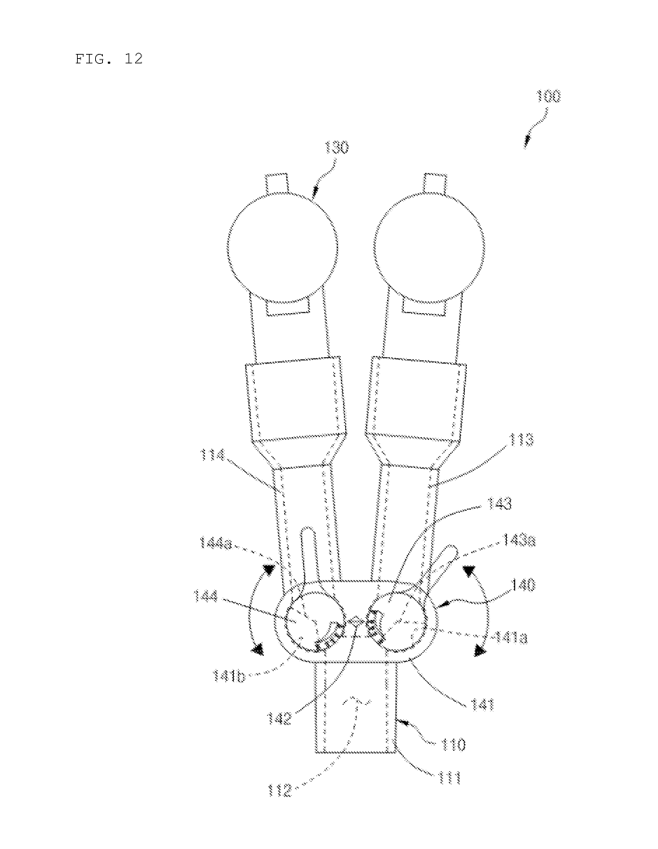

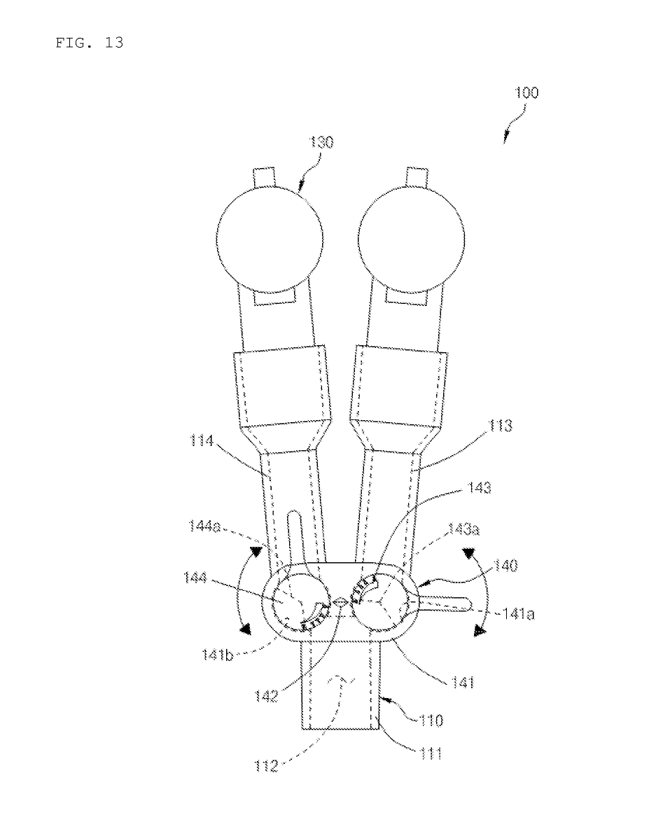

FIG. 9 is a perspective view of a connector of a double-lumen tube for DLV according to another embodiment of the present invention, FIG. 10 is a right side view of the connector of a double-lumen tube for DLV illustrated in FIG. 9, FIG. 11 is a view illustrating a use state with a connection valve unit completely open in the connector of a double-lumen tube for DLV illustrated in FIG. 9, FIG. 12 is a view illustrating a use state with a part of the connection valve unit closed, as a first rotation valve is operated in the use state of the connector of a double-lumen tube for DLV illustrated in FIG. 11, and FIG. 13 is a view illustrating a use state with the connection valve unit completely closed, as the first rotation valve is operated in the use state of the connector of a double-lumen tube for DLV illustrated in FIG. 12.

Referring to FIGS. 9 to 12, the connector 100 of a double-lumen tube for DLV according to another embodiment of the present invention includes the tube connection unit 110, a connection valve unit 140, and the ventilator connection unit 130. The ventilator connection unit 130 is identical to its counterpart in the connector 100 of a double-lumen tube for DLV illustrated in FIGS. 1 to 5 and thus will not be described herein. The following description centers on the connection valve unit 120 different from in the connector 100 of a double-lumen tube for DLV illustrated in FIGS. 1 to 5.

The tube connection unit 110 includes the tube connection body 111 into which the double-lumen tube is inserted, the first branched pipe 113, and the second branched pipe 114. The tube connection body 111 is disposed at one side of the double-lumen tube. The tube connection space 112 branched through the connection valve unit is formed in a portion of the tube connection body 111 and a pair of lumen tubes are inserted into the other portion of the tube connection body 111. The tube connection space 112 is formed in the tube connection body 111. The pair of lumen tubes are inserted into the other portion of the tube connection space 112 and one portion of the tube connection space 112 is branched in both directions so that the pair of lumen tubes may be connected to the ventilator connection unit 130. As the connection valve unit 140 is installed at positions where the tube connection space 112 is branched in both directions in order to control the flow amount of gas or fluid and provide or block pressure, the connection valve unit 140 controls the flow amount and pressure at a position where fluid and pressure are directly provided. Therefore, control efficiency can be increased.

The first branched pipe 113 is disposed at one side of the tube connection body 111. The first branched pipe 113 is installed at a branched position of the connection valve unit 140, for connection to one of the pair of lumen tubes, when the double-lumen tube is connected to the connector 100. That is, the first branched pipe 113 is provided to connect one of the lumen tubes inserted into the other portion of the tube connection space 112 to the ventilator connection unit 130, with the flow amount controlled by the connection valve unit 140.

The second branched pipe 114 is disposed at the one side of the tube connection body 111. The second branched pipe 114 is installed at a branched position of the connection valve unit 140, for connection to the other lumen tube, when the double-lumen tube is connected to the connector 100. That is, the second branched pipe 114 is provided to connect the other lumen tube inserted into the other portion of the tube connection space 112 to the ventilator connection unit 130, with the flow amount controlled by the connection valve unit 140.

The connection valve unit 140 includes a connection valve body 141 installed in the tube connection body 111, a reference position indicator 142, a first rotation valve 143, and a second rotation valve 144.

The connection valve body 141 is installed in one direction in which the tube connection body 111 is branched. The connection valve body 141 is installed at branched positions of the tube connection space 112 and includes a first valve space 141a communicating with a branched position for connection to the one lumen tube and a second valve space 141b communicating with a branched position for connection to the other lumen tube. The first valve space 141a is formed at one branched portion of the tube connection space 112 so that the one lumen tube may be connected to the first branched pipe 113 inside the connection valve body 141. The second valve space 141b is formed at another branched portion of the tube connection space 112 so that the other lumen tube may be connected to the second branched pipe 114 inside the connection valve body 141.

That is, the connection valve body 141 forms the first valve space 141a at a branched position connected to the first branched pipe 113 and the second valve space 141b at a branched position connected to the second branched pipe 114 inside the connection valve body 141 that is branched.

The reference position indicator 142 is provided at a center of the connection valve body 141 and includes protruding scales between the first and second valve spaces 141a and 141b. The reference position indicator 142 is configured to allow setting of a reference position, for flow control in the first and second valve spaces 141a and 141b. Thus, the reference position indicator 142 provides a reference for controlling to an accurate position upon receipt of an external instruction and indicates transfer or blocking of pressure and a controlled flow amount, thereby improving the stability of flow control.

The first rotation valve 143 is disposed in one direction from the center of the connection valve body 141, rotatably in the first valve space 141a. As the first rotation valve 143 is rotated, it controls the flow amount of gas or fluid provided from the ventilator connection unit 130 so that the gas or fluid may be supplied to the one lumen tube.

The first rotation valve 143 includes a first circulation space 143a at a position connected to the one lumen tube, for circulating the gas or fluid provided by the ventilator. As an area over which the first circulation space 143a is opened and closed by rotation of the first rotation valve 143, the flow amount for the one lumen tube is controlled.

A first valve knob 143b protrudes in one direction of the first rotation valve 143 so that the user may grab the first valve knob 143b. The user grabs the first valve knob 143b and rotates it so as to control an area over which the first circulation space 143a is opened or closed.

A first position indicator 143c is provided in the other direction of the first rotation valve 143, including a plurality of scales directed toward the reference position indicator 142, apart from each other by a predetermined distance, in order to indicate a flow control position according to a degree to which the first circulation space 143a is opened or closed.

That is, if the first rotation valve 143 is operated to control the flow amount of the one lumen tube, the user grabs and rotates the first valve knob 143b to an accurate position, while observing the first position indicator 143c with respect to the reference position indicator 142. Thus the area of the first circulation space 143a is controlled suitably according to the controlled flow amount and the resulting accurate flow control increases stability, while the controlled flow amount is indicated. Therefore, as accurate flow control is enabled, accuracy can be increased.

The second rotation valve 144 is disposed in the other direction from the center of the connection valve body 141, rotatably in the second valve space 141b. As the second rotation valve 144 is rotated, it controls the flow amount of gas or fluid provided from the ventilator connection unit 130 so that the gas or fluid may be supplied to the other lumen tube.

The second rotation valve 144 includes a second circulation space 144a at a position connected to the other lumen tube, for circulating the gas or fluid provided by the ventilator. As an area over which the second circulation space 144a is opened and closed by rotation of the second rotation valve 144, the flow amount for the other lumen tube is controlled.

A second valve knob 144b protrudes in one direction of the second rotation valve 144 so that the user may grab the second valve knob 144b. The user grabs the second valve knob 144b and rotates it so as to control an area over which the second circulation space 144a is opened or closed.

A second position indicator 144c is provided in the other direction of the second rotation valve 143, including a plurality of scales directed toward the reference position indicator 142, apart from each other by a predetermined distance, in order to indicate a flow control position according to a degree to which the second circulation space 144a is opened or closed.

That is, if the second rotation valve 144 is operated to control the flow amount of the other lumen tube, the user grabs and rotates the second valve knob 144b to an accurate position, while observing the second position indicator 144c with respect to the reference position indicator 142. Thus the area of the second circulation space 144a is controlled suitably according to the controlled flow amount and the resulting accurate flow control increases stability, while the controlled flow amount is indicated. Therefore, as accurate flow control is enabled, accuracy can be increased.

As described before, the first and second rotation valves 143 and 144 are installed at both portions of the connection valve body 144 installed at branched positions of the tube connection body 111, to which a pair of tubes are connected. Thus, when flow control is performed by rotating the first and second rotation valves 143 and 144, a flow control degree is indicated. Accordingly, the stability and accuracy of flow control can be increased.

As is apparent from the foregoing description, a connector of a double-lumen tube for DLV according to an embodiment of the present invention, which connects a double-lumen tube intubated into the lungs to apply pressure to the lungs to a ventilator for providing pressure, is configured so as to enable immediate pressure transfer or blocking through a user manipulation and enable flow control. Therefore, the stability of DLV and manipulation convenience can be increased.

Since the connector of a double-lumen tube for DLV according to the present invention is connected to each of a pair of lumen tubes, transfers or blocks pressure to each lumen tube, and allows a user's physical manipulation, the pressure and flow amount of supplied gas or fluid can be controlled according to a lung state and thus surgical safety can be increased.

Further, the connector of a double-lumen tube for DLV according to the present invention includes valves connected to the pair of lumen tubes inserted into the respective lungs, for transferring or blocking pressure to the lungs and controlling a flow amount for the lungs, and an indicator for indicating an operation state and a flow control. Therefore, even a non-skilled operator can readily perform a flow control, continuously monitor the amount of externally supplied gas or fluid, and take an immediate action. Accordingly, surgical safety and manipulation convenience can be increased.

Those skilled in the art will appreciate that the present invention may be carried out in other specific ways than those set forth herein without departing from the spirit and essential characteristics of the present invention. The above embodiments are therefore to be construed in all aspects as illustrative and not restrictive. The scope of the invention should be determined by the appended claims and their legal equivalents, not by the above description, and all changes coming within the meaning and equivalency range of the appended claims are intended to be embraced therein.

What is claimed is:

1. A connector of a double-lumen tube for Differential Lung Ventilation (DLV), for connecting a pair of lumen tubes of the double-lumen tube intubated respectively into both bronchi through an airway to a ventilator that provides pressure, the connector comprising:
a tube connection unit disposed at one side of the double-lumen tube, and branched in one direction to connect to the ventilator, with the pair of lumen tubes of the double-lumen tube inserted into the tube connection unit;
a connection valve unit disposed in the one direction in which the tube connection unit is branched, for controlling a flow amount of gas or fluid provided to the respective branched lumen tubes and externally indicating an operation state of a valve that controls the flow amount of the gas or the fluid; and
a ventilator connection unit disposed at one side of the connection valve unit, in connection to the ventilator, for providing pressure of the ventilator separately to the pair of lumen tubes through the connection valve unit.

2. The connector according to claim 1, wherein the tube connection unit comprises:
a tube connection body disposed at one side of the double-lumen tube and having one portion branched through the connection valve unit and the other portion into which the double-lumen tube is inserted;
a first branched pipe disposed at one side of the tube connection body and installed at a position branched through the connection valve unit, for connection to one of the pair of lumen tubes when the double-lumen tube is connected; and
a second branched pipe disposed at the one side of the tube connection body and installed at a position branched through the connection valve unit, for connection to the other lumen tube when the double-lumen tube is connected.

3. The connector according to claim 1, wherein the connection valve unit comprises:
a connection valve body installed in the one direction in which the tube connection unit is branched and including a first valve space communicating with one of the pair of lumen tubes, at a branched position of the tube connection unit and a second valve space communicating with the other lumen tube, at a branched position of the tube connection unit;
a first rotation valve disposed in one direction from a center of the connection valve body, installed rotatably in the first valve space, and including a first circulation space at a position connected to the one lumen tube, for circulating the gas or the fluid supplied from the ventilator and a first valve knob protruding in one direction to allow a user to grab the first valve knob, for controlling the flow amount of the gas or the fluid to the one lumen tube by controlling an area over which the first circulation space is opened and closed; and
a second rotation valve disposed in the other direction from the center of the connection valve body, installed rotatably in the second valve space, and including a second circulation space at a position connected to the other lumen tube, for circulating the gas or the fluid supplied from the ventilator and a second valve knob protruding in one direction to allow the user to grab the second valve knob, for controlling the flow amount of the gas or the fluid to the other lumen tube by controlling an area over which the second circulation space is opened and closed.

4. The connector according to claim 3, further comprising:
a reference position indicator provided at the center of the connection valve body and including protruding scales between the first and second rotational valves, for indicating a reference position for adjusting the first and second rotational valves that rotate;
a first position indicator disposed at a position of the second rotational valve in the other direction from the first rotation valve, with a plurality of scales directed toward the reference position indicator, the scales apart from one another by a predetermined distance, for indicating a position to which the flow amount of the gas or the fluid is controlled with respect to the reference position indicator according to a degree of opening or closing of the first circulation space; and
a second position indicator disposed at a position of the first rotation valve in one direction from the second rotation valve, with a plurality of scales directed toward the reference position indicator, the scales apart from one another by a predetermined distance, for indicating a position to which the flow amount of the gas or the fluid is controlled with respect to the reference position indicator according to a degree of opening or closing of the second circulation space.

5. The connector according to claim 1, wherein the ventilator connection unit comprises:
a first ventilator connection body disposed at the one side of the connection valve unit connected to one of the pair of lumen tubes and having one lateral end connected to the ventilator at a lateral position;
a first connection body disposed at the other side of the first ventilator connection body, for connecting the first ventilator connection body to the connection valve unit connected to the one lumen tube;
a second ventilator connection body disposed at the one side of the connection valve unit connected to the other lumen tube and having one lateral end connected to the ventilator at a lateral position; and
a second connection body disposed at the other side of the second ventilator connection body, for connecting the second ventilator connection body to the connection valve unit connected to the other lumen tube.

6. The connector according to claim 5, further comprising:
a first drain body disposed at the other lateral end of the first ventilator connection body, for opening or closing the other lateral end of the first ventilator connection body to drain internal pressure through a user manipulation; and
a second drain body disposed at the other lateral end of the second ventilator connection body, for opening or closing the other lateral end of the second ventilator connection body to drain internal pressure through a user manipulation.
